(19) United States
(12) Patent Application Publication
(10) Pub. No.: US 2026/0055377 A1
(43) Pub. Date: Feb. 26, 2026

(54) METHODS FOR CULTURING AND DIFFERENTIATING PLURIPOTENT CELLS INTO PROGENITOR OR MATURE MUSCLE CELLS AND COMPOSITION COMPRISING SAID CELLS

(71) Applicant: RAMOT AT TEL AVIV UNIVERSITY LTD., Tel Aviv (IL)

(72) Inventors: Iftach NACHMAN, Tel Aviv (IL); Gaya SAVYON, Tel Aviv (IL)

(21) Appl. No.: 19/372,381

(22) Filed: Oct. 29, 2025

Related U.S. Application Data
(63) Continuation-in-part of application No. 18/278,100, now Pat. No. 12,467,036.
(60) Provisional application No. 63/164,047, filed on Mar. 22, 2021.

(51) Int. Cl. C12N 5/071

(52) U.S. Cl. CPC C12N 5/0697; C12N 2501/105; C12N 2501/115; C12N 2501/12; C12N 2501/155; C12N 2501/415; C12N 2506/02; C12N 2513/00

(57) ABSTRACT

Provided herein are artificially cultured somites comprising mature muscle progenitor cells and/or mature muscle cells, methods of obtaining same and methods for fast, large scale production of cultured meat comprising mature muscle cells.

This application is a Continuation-In-Part application of U.S. application Ser. No. 18/278,100 filed on Aug. 21, 2023, which is a National Phase Application under 35 U.S.C. § 371 of International Application No. PCT/IL2022/050310 filed on Mar. 20, 2022, designating the U.S. and published as WO 2022/201147 on Sep. 29, 2022, which claims the benefit of U.S. Provisional Application No. 63/164,047 filed Mar. 22, 2021. Any and all applications for which a foreign or domestic priority claim is identified above and/or in the Application Data Sheet as filed with the present application are hereby incorporated by reference under 37 CFR 1.57.

### FIELD OF THE INVENTION

Provided herein are artificially cultured somites including mature muscle progenitor cells and/or mature muscle cells, methods of obtaining same and methods for fast, large scale production of cultured meat including mature muscle cells.

### BACKGROUND OF THE INVENTION

The meat industry is one of the main causes of environmental and climate degradation. Due to world population growth and increased demand, meat consumption is expected to double between 1999 and 2050. This industry is also heavily associated with the onset of antibiotic resistant and zoonotic diseases. Although a completely plant-based diet can be considered a good alternative to reduce the environmental impact, there is much reason to believe that such a diet will not be adapted voluntarily by most of the world population while meat is still available. Cultured meat is produced in-vitro using cell cultures, offering an alternative production method for meat that is both cheap, more ethical and environmentally friendly, but at the same time keeps the characteristic taste, texture and nutritional value, and even the same biological origin as the animal-derived products. Since skeletal muscles are the main edible tissue used in most animal meat, generation of skeletal muscles in large mass in-vitro is required for the efficient production of cultured meat.

Schmidt et al. (Cellular and Molecular Life Sciences, 76:2559-2570, 2019) discloses regeneration of skeletal muscle from satellite cells, the stem cells of skeletal muscle.

Suzuki et al. (Differentiation, 96:70-81, 2017) discloses protocol for the derivation of myogenic progenitors directly (without genetic modification) from human pluripotent cells using free-floating spherical culture through a long differentiation period of over 6 weeks, in culture medium supplemented with 100 ng/ml FGF-2 and 100 ng/ml EGF.

Nachman et al. (bioRxiv, 728642, 2019) disclose that the basic rules governing definitive endoderm cells onset from within the mesendoderm progenitors population, involve self-sorting of Sox17+ cells preceded by up-regulation of E-cadherin, wherein a small subpopulation of high-expressing Bra cells are committed to their Sox17+ fate independently of external Wnt signal.

There is an unmet need for efficient, fast and cost-effective process for producing in-vitro muscle cells, preferably within a tissue structure.

### SUMMARY OF THE INVENTION

Provided herein is a developmental-path based serum-free, genomic modifications free, protocol for the generation, in culture, of somites, myogenic progenitors, muscle cells and muscle tissue. The protocol includes inducing muscle differentiation within 3D suspended cell cultures, starting from embryonic stem cells. Further provided are cultured somites and somite-like structures including muscle cells.

Advantageously, the protocol disclosed herein for the preparation of meat products in culture, combines the self-organization properties of embryonic 3D developmental system at the micro scale, with biotechnological solutions, which render it highly suitable for mass production of meat products and for muscle tissue maturation within organoid cultures. The protocol cures the large gap between existing established muscle differentiation protocols and the scaleup and safety requirements for food production.

Surprisingly, the protocol disclosed herein produces somites, and somite-like structures, which are enclosed structures that give rise to vertebrae and skeletal muscle in the embryo. The production of somites has major benefits. First, production of somite indicates that the protocol (method/process) disclosed herein well mimics the developmental stages that cells undergo during embryonic development from pluripotent (ES) cells to fully differentiated skeletal muscle cells, in a 3D suspension organoid format. As a result, the cultured tissue produced herein includes all the feature of ‘real’ meat, namely, it is primarily made of muscle tissue but also includes adipose cell, connective tissue, and blood, contributing to the structure and taste of meat produced from livestock. Second, the presence of somites in the production process results in intra-organoid signaling, which promotes the required differentiation independently of external supplements, and hence only little amounts of growth factors are used for supplementing the culture medium (for example, in the order of about 10 ng/ml). Accordingly, the protocol disclosed herein is cost-effective, since it does not require large quantities of costly growth factors (that typically create the major expenditure in the production of cultured tissues).

Unexpectedly, applying the protocol disclosed herein on pluripotent cells results with the production of myogenic progenitors in a short time frame, for example, in only two weeks, using no more than about 20 ng/ml FGF2 and EGF growth factors.

Moreover, as the developmental process, including somites formation and muscle formation, is highly preserved among vertebrates—the protocol disclosed herein which follows embryonic development guidelines, can be successfully implemented to produce cultured meat from progenitor cells of various species.

Finally, the present protocol initiates with stem cell cells and enables differentiation in suspension (e.g. in large scale bioreactors or even in conventional steerers), until obtaining myogenic progenitors.

According to some embodiments, in view of the above, the protocol disclosed herein being fast, cost-effective, reproducible, suitable for large scale bioreactors, and yet producing cultured meat that includes the feature of livestock meat, is highly appropriate for industrial manufacturing of cultured myogenic progenitors and cultured meat.

The disclosed protocol is composed of a series of steps, where each step attempts to efficiently mimic the in-vivo signaling environment sensed by the myogenic lineage at the relevant developmental stage, e.g. mesoderm, presomitic mesoderm, myogenic progenitors and mature muscle cells. The first step of the protocol includes aggregating mES cells under a first medium, following the differentiation of the aggregate in suspension, where the medium may be modified between some consecutive steps, as further detailed and exemplified hereinbelow.

There is provided, according to some embodiments, a method for generating somites, the method including:
(a) suspending embryonic stem cells in culture medium to obtain essentially spheric aggregates;
(b) adding to the culture medium at least one Wnt activator to obtain aggregates including mesodermal and endodermal cells;
(c) removing the Wnt activator;
(d) observing the aggregates including the mesodermal and endodermal cells, and adding extracellular matrix or components thereof after identifying mesodermal polarization in the aggregates, to obtain aggregates including paraxial mesoderm; and
(e) incubating the aggregates of step (d), for 10 to 48 hours, thereby obtaining aggregates including a plurality of somites.

According to some embodiments, the method further includes adding to the culture medium of step (a) one or more of insulin, Knock-out Serum Replacement, transferrin-selenium and BMP4.

According to some embodiments, the Wnt activator is selected from the group consisting of: Chir99021, Wnt3a and Rspo3.

According to some embodiments, adding to the culture medium at least one Wnt activator is carried out within 12 to 36 hours after obtaining said essentially spheric aggregates.

According to some embodiments, removing the Wnt activator is carried out within 8 to 36 hours after the addition thereof. According to some embodiments, removing the Wnt activator is carried out within 12 to 36 hours after the addition thereof.

According to some embodiments, the method may further include adding at least one Nodal inhibitor to the culture medium following appearance of the aggregates including said mesodermal and endodermal cells.

According to some embodiments, the amount of the extracellular matrix or components thereof is within the range of about 1 to 15% vol/vol.

According to some embodiments, the mesodermal polarization is identified by the aggregates assuming an ovoid morphology as determined by an aspect ratio of the aggregates being 1.2 or higher.

According to some embodiments, the mesodermal polarization is identified by staining mesodermal or presomitic mesoderm (PSM) cells in the aggregates including the mesodermal and endodermal cells with a suitable marker, to identify the mesodermal or PSM cells accumulating at poles of the aggregates.

According to some embodiments, the embryonic stem cells are from human, mouse, or fish.

According to some embodiments, the embryonic stem cells are from teleost fish. According to some embodiments, the embryonic stem cells are from seabream or eel. According to some embodiments, the embryonic stem cells are from seabream.

According to some embodiments, the method may further include adding in step (d) at least one compound selected from the group consisting of: Wnt activator and BMP inhibitor.

According to some embodiments, the method may further include removing the extracellular matrix or components thereof, following obtaining the plurality of somites.

According to some embodiments, the method further includes a step of adding one or more growth factors to the culture medium in step (e), the concentration of the one or more growth factors being below 30 ng/ml, thereby generating myogenic progenitors.

According to some embodiments, there is provided method for generating myogenic progenitors, the method including:
(a) suspending embryonic stem cells in culture medium to obtain spheric aggregates;
(b) adding to the culture medium at least one Wnt activator to obtain aggregates including mesodermal and endodermal cells;
(c) removing the Wnt activator;
(d) observing the aggregates including the mesodermal and endodermal cells, and adding extracellular matrix or components thereof after identifying mesodermal polarization in the aggregates, to obtain aggregates including paraxial mesoderm;
(e) incubating the aggregates of step (d), for 10 to 48 hours, thereby obtaining aggregates including a plurality of somites; and
(f) adding one or more growth factors to the medium in step (e), the concentration of the one or more growth factors being below 30 ng/ml, thereby obtaining myogenic progenitors.

According to some embodiments, the one or more growth factors may include HGF, IGF and FGF2.

According to some embodiments, step (a) may be carried out in suspension.

According to some embodiments, the method may further include adding to the medium of step (d) at least one of compound selected from: BMP inhibitor and Wnt activator. According to some embodiments, the Wnt activator is selected from the group consisting of: Chir99021, Rspo3 and Wnt3a.

According to some embodiments, adding to the culture medium at least one Wnt activator is carried out within 12 to 36 hours after obtaining said spheric aggregates.

According to some embodiments, the method may further include removal of the extracellular matrix (ECM) or components thereof prior to step (e). According to some embodiments, the method may further include removal of the extracellular matrix or components thereof prior to step (f).

According to some embodiments, the concentration of each growth factor is within the range of about 1 to 25 ng/ml.

According to some embodiments, the one or more growth factors is IGF, and the concentration of IGF is within the range of about 1 to 5 ng/ml.

According to some embodiments, the myogenic progenitors include myoblasts and myocytes.

According to some embodiments, the method may further include adding to the culture medium of step (a) one or more of insulin, Knock-out Serum Replacement, transferrin-selenium and BMP4.

According to some embodiments, removing the Wnt activator may be carried out within the range of about 8 to 36 hours. According to some embodiments, removing the Wnt activator may be carried out within about 12 to 36 hours after the addition thereof.

According to some embodiments, the method may further include adding at least one Nodal inhibitor to the culture medium following appearance of the aggregates including said mesodermal and endodermal cells.

According to some embodiments, the amount of the extracellular matrix or components thereof may be within the range of about 1 to 15% vol/vol.

According to some embodiments, there are provided cultured somites which include mature muscle progenitor cells and/or mature muscle cells, prepare by the methods disclosed herein.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1A shows an aggregate containing mesodermal cells (Brachyury-positive cells (Bra-GFP); top panel) and endodermal cells (Sox17-positive cells (Sox-RFP); middle panel), obtained from ESC cells seeded in plates following Step 2 in the disclosed protocol.

FIG. 1B shows aggregates containing mesodermal cells (Brachyury (an early mesoderm marker)-positive cells; narrow arrows) and endodermal cells (Sox17-positive cells; thick white arrows), obtained from ESC cells in suspension, following Step 2 in the disclosed protocol.

FIG. 2A represents posterior presomitic mesoderm (pPSM) spatio-temporal pattern in mouse 3D organoids formed during Step 3 of the disclosed protocol. The organoids are aggregated from Msgn1-YFP mES cells and cultured for 6 days in suspension. Scale bar 100 μm.

FIG. 2B represents the fluorescence level plotted against absolute organoid length (left panel) and against normalized organoid length (right panel) measured for 20 organoids formed during Step 3 of the disclosed protocol. Msgn1-YFP marks pPSM.

FIG. 2C represents a two-cluster elongated organoid: Msgn1+ cells migrate to form two clusters.

FIG. 2D represents a three-cluster organoid: Msgn1+ cells migrate to form three clusters, all extending from the organoid's core.

FIG. 2E represents a histogram of the number of Msgn1+ clusters per organoid.

FIG. 3A represents Day-5 organoid formed during Step 3, fixed and immunostained for Brachyury (located on the left/posterior pole, marked by grey arrow), where Msgn1-YFP marks pPSM (bright regions, marked by dashed arrows).

FIG. 3B is a schematic description of the organoid shown in FIG. 3A, including Brachyury-positive cells at the posterior pole, Msgn1-positive pPSM cells and anterior presomitic mesoderm (aPSM) cells at the anterior pole.

FIG. 3C represents delta CT mean of pPSM associated genes on Day 4 (black columns) and Day 6 (grey columns) of differentiation of mESC derived organoids towards PSM.

FIG. 3D represents exemplary images of Msgn1-YFP mESC organoids (middle panel) immunostained for Pax3 (right panel), on Day 6 of differentiation. Scale bar 100 μm.

FIG. 3E represents exemplary images of Msgn1-YFP mESC organoids (middle panel) immunostained for N-cadherin (Cdh2; right panel), on Day 6 of differentiation. Scale bar 100 μm.

FIGS. 4A-4C represent images of aggregates obtained during Step 3. Msgn1-YFP (bright regions) indicates the presence of pPSM. Scale bar 100 μm.

FIGS. 5A-5C represent images of an elongated aggregate obtained at the end of Step 4, stained with Somites marker (Pax3) and imaged at two z-sections (left, center) and brightfield (right). Somites are numbered. Thick white arrows point at the Sox17-RFP positive region, marking endoderm. Scale bar 100 μm.

FIG. 5D represents an image of an elongated aggregate obtained at the end of Step 4, with Brachyury-GFP and Sox17-RFP positive regions, marking mesoderm and endoderm, respectively. Somites are indicated by small white arrows pointing circular structures at the anterior region. Scale bar 100 μm.

FIG. 6A represents image of aggregates obtained at the end of Step 5, stained with myogenic progenitor marker-Pax7 (bright regions at the upper half of the aggregate) and Sox17-RFP positive endoderm region (bottom left, marked by white arrows). Scale bar 100 μm.

FIG. 6B represents image of aggregates obtained at the end of Step 5, stained with myoblasts marker—MyoD (bright regions). Scale bar 100 μm.

FIG. 6C represents image of aggregates obtained at the end of Step 5, stained with myocytes marker—MyoG (bright regions). Scale bar 100 μm.

FIG. 6D represents image of mature somites, where black arrows point at representative somites. Scale bar 100 μm.

FIG. 7A shows aggregate surface area between day 1 and day 13 under different pulse conditions.

FIG. 7B shows phalloidin rhodamine staining (red) 24 days after seeding, demonstrating fiber-like morphology, indicating skeletal muscle formation.

FIG. 7C shows titin staining (green) of two-month-old aggregates indicating muscle differentiation.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

While cell-based meat science has evolved in the past few years, obtaining a large mass of skeletal muscle tissue, is a major challenge. For this to be met, a scalable and efficient proliferation and muscle differentiation process is needed. While suspension culture methods offer the greatest promise for scaleup, they pose a hurdle toward muscle differentiation, due to the current need in physical anchoring points.

In recent years, tremendous advances have been made in 3D suspension culture of small organ-like 3D structures (termed organoids), that mimic various aspects of the original organ, including cell composition. Many of the organoid systems start from embryonic stem cells, offering a complete start-to-finish differentiation process, mimicking the embryonic process to some extent, with no need to repeatedly harvest the starting cell population from animals.

In brief, there are two main approaches for producing cultured meat: (1) starting from satellite cells—the relative benefit of cultured satellite cells is that their differentiation to muscle cells is comparatively fast and easy. However, as satellite cells are post mitotic (terminally differentiated) they do not proliferate and hence their use as the starting cells is not feasible for mass production; (2) starting from pluripotent cells (embryonic stem cells)—the benefit of using pluripotent cell is their outstanding long-term proliferative potential and their ability to be driven towards differentiate, upon being exposed to suitable conditions. However, the differentiation process of these cells to mature muscle cells takes very long time (over a month, usually, over 6 weeks).

A major challenge is the efficient growth of large mass of muscle cells, preferably within a tissue context, namely, cultured meat that resembles the contents of meat produce which is mainly composed of muscle tissue (about 90%) and further includes a combination of adipose (fat) cells, blood and connective tissue. The current solutions mostly rely on adult progenitor cells (e.g. satellite cells) as a starting point, and single cell type differentiation protocols, limiting both the proliferation capacity and the resemblance to full tissue structure. A scalable and efficient proliferation and muscle differentiation process is therefore required.

According to some embodiments, utilizing the protocol as disclosed herein allows producing cultured meat that includes the various component of meat produce, primarily since the protocol produces somatic cells.

The term “protocol” as used herein is interchangeable with the term “method” or “process” in the context of a protocol for obtaining somites and 3D edible organoids.

Muscle differentiation protocols known to date are either based on 2D adherent cultures, or are not efficient. Many protocols use serum and/or genetic modifications to activate certain key genes. Some use partially differentiated cells (such as satellite cells) as the starting cell type, which may place limits on the proliferation rate and capacity of these starting cells. The protocol disclosed herein combines signaling-only differentiation protocols, with suspended 3D culturing systems (starting from ES cells) resulting in an efficient muscle differentiation method which is scalable and free from serum and other animal-derived factors. The use of pluripotent stem cells removes the need to repeatedly harvest the starting cell population from animals.

Thus, according to some embodiments, there is provided a method for generating somites, the method including:
(a) suspending embryonic stem cells in culture medium to obtain essentially or substantially spheric aggregates;
(b) adding to the culture medium at least one Wnt activator to obtain aggregates including mesodermal and endodermal cells;
(c) removing the Wnt activator;
(d) observing the aggregates which includes the mesodermal and endodermal cells, and adding extracellular matrix (ECM) or components thereof after identifying mesodermal polarization in the aggregates, to obtain aggregates which include paraxial mesoderm; and
(e) incubating the aggregates of step (d), for 10 to 48 hours, thereby obtaining aggregates including/having a plurality of somites.

According to some embodiments, the term “suspending” as used herein refers to culturing in suspension (3D) under conditions that are suitable for maintaining proliferation and pluripotency of the pluripotent cells. Cell proliferation generally increases the size of the aggregates forming larger aggregates, which can be routinely mechanically or enzymatically dissociated into smaller aggregates to maintain cell proliferation within the culture and increase numbers of cells. Commonly, cells cultured within aggregates in maintenance culture maintain markers of pluripotency. Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages. The pluripotent stem cell aggregates require further differentiation cues to induce differentiation.

According to some embodiments, at the end of step (a) of the method, a plurality of spheric aggregates are obtained. According to some embodiments, the plurality of spheric aggregates obtained at the end of step (a) include at least 50 spheric aggregates, at least 100 spheric aggregates, at least 150 spheric aggregates, at least 200 spheric aggregates, at least 250 spheric aggregates, at least 300 spheric aggregates, about 300 spheric aggregates. Each possibility represents a separate embodiment.

As used herein, the term “about” may be used to specify a value of a quantity or parameter (e.g. amount, percentage, length) within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

The terms “pluripotent stem cell”, “embryonic stem cell” and ESC as used herein are interchangeable.

According to some embodiments, the embryonic stem cells are derived from livestock. According to some embodiments, the embryonic stem cells are derived from animals selected from the groups consisting of fish, cattle, sheep, goats, swine and poultry. According to some embodiments, the embryonic stem cells are derived from fish, mouse, and human. According to some embodiments, the embryonic stem cells are derived from fish.

The term “fish”, as used herein, relates to a cold-blooded, aquatic vertebrate animal having gills and fins.

According to some embodiment, the fish is a bony fish. According to some embodiment, the fish is a teleost.

A teleost is a member of the infraclass Teleostei. Teleosts are characterized by several anatomical features, most notably: movable premaxillae in the jaw, allowing them to protrude their jaws outward to efficiently capture prey; homocercal caudal fin, meaning the upper and lower lobes of the tail fin are about equal in size; and unpaired basibranchial tooth plates in the pharyngeal jaws, which are a second set of jaws located in the throat.

Nonlimiting examples of teleosts include seabream, eels, catfish, tarpon, tuna, halibut, flounder, trout, cod, herring, salmon, anglerfish, and seahorses.

According to some embodiments, the teleost belong to the Sparidae family. According to some embodiments, the teleost is a seabream or an eel. According to some embodiments, the teleost is a seabream. According to some embodiments, the seabream is gilthead seabream (Sparus aurata).

It is to be understood, that the embryonic stem cell (ESC) used herein are free of genetic engineering.

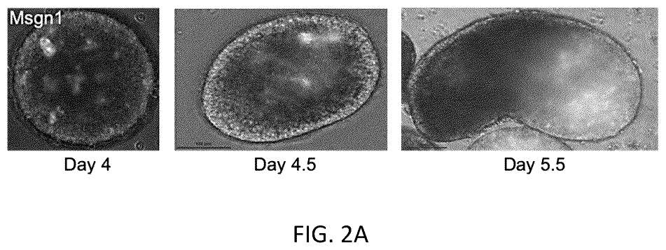

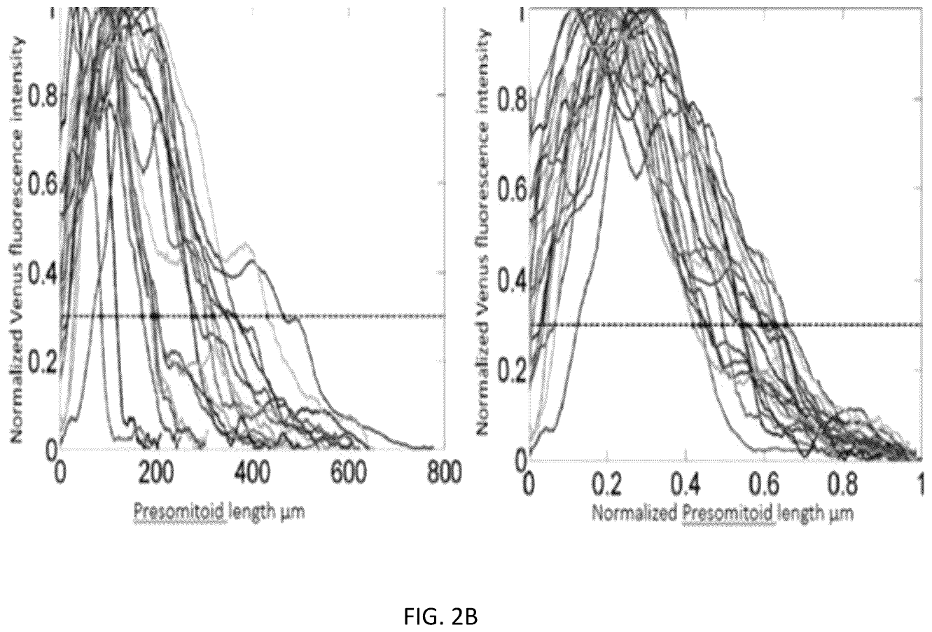

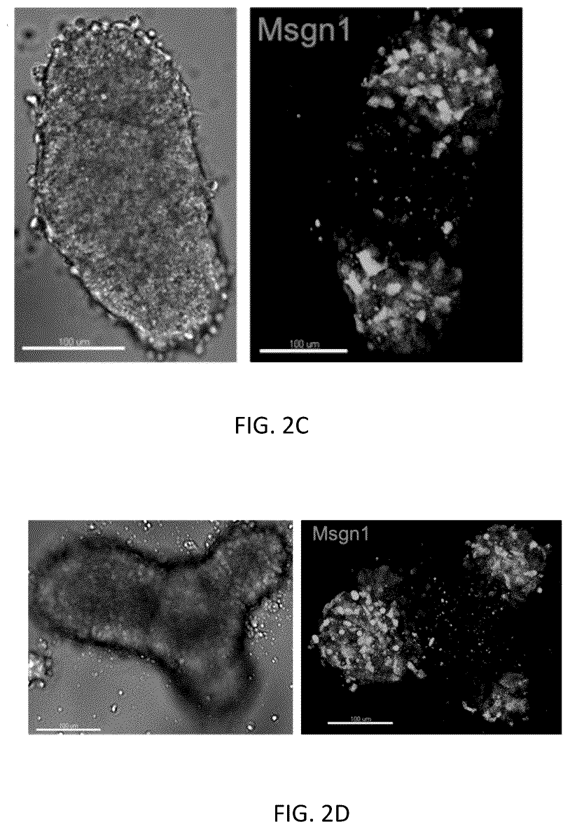

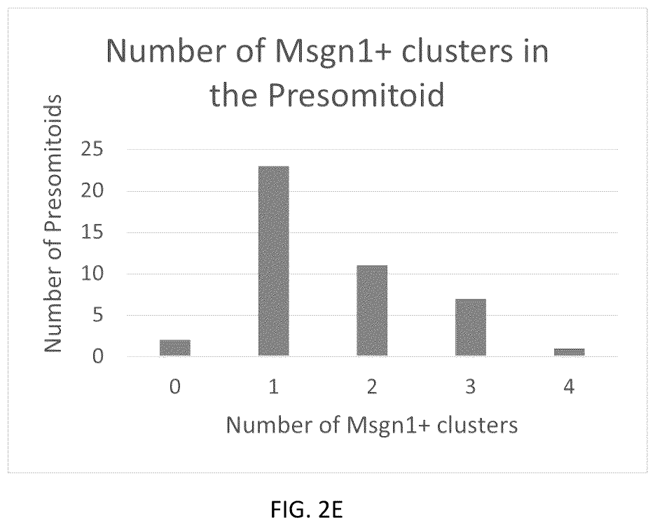

Pluripotent stem cell morphology has classical morphological features of an embryonic stem cell. Normal embryonic stem cell morphology is characterized by being round (spheric) and small in shape, with a high nucleus-to-cytoplasm ratio, the notable presence of nucleoli, and typical inter-cell spacing. In contrast, aggregates including mesodermal and endodermal cells, as obtained by step (b) of the method disclosed herein have ovoid morphology, as demonstrated hereinbelow, for example, as shown in FIG. 2A, day 4.5.

According to some embodiments, step (a) is carried out on culture plates (e.g. Petri dishes and/or multi-well plates). Thus, according to some embodiments, the embryonic stem cells form an adherent cell culture, in culture plate(s). According to some embodiments, the method may further include adding to the culture medium of step (a) one or more of insulin, transferrin-selenium, Knock-out Serum Replacement and BMP4. Each possibility is a separate embodiment.

The step of adding to the culture medium of step (a) one or more of insulin, transferrin-selenium, Knock-out Serum Replacement and BMP4 is carried out in order to increase the fraction of presomitic mesoderm (PSM) cells within the organoids/aggregates. Hence, supplementing the culture medium of step (a) with insulin, transferrin-selenium, Knock-out Serum Replacement and/or BMP4 improves the quality of the protocol disclosed herein.

According to some embodiments, insulin may be added to the culture medium. According to some embodiments, the concentration of insulin added to the culture medium is within the range of 5 to 15 μl/ml, 5 to 13 μl/ml, 7 to 13 μl/ml, 7 to 12 μl/ml, 5 to 12 μl/ml, about 7 μl/ml, or about 10 μl/ml. Each possibility represents a separate embodiment.

According to some embodiments, transferrin-selenium may be added to the culture medium. According to some embodiments, the concentration of transferrin-selenium added to the culture medium is within the range of 0.1% to 5%, 0.2% to 4%, 0.2% to 3%, 0.5% to 3%, 0.5% to 2%, 0.8% to 3%, 0.8% to 2%, about 2%, about 1.5% or about 1%. Each possibility represents a separate embodiment.

According to some embodiments, BMP4 may be added to the culture medium. According to some embodiments, the concentration of BMP4 added to the culture medium is within the range of 5 to 15 μl/ml, 5 to 13 μl/ml, 7 to 13 μl/ml, 7 to 12 μl/ml, 5 to 12 μl/ml, about 7 μl/ml, or about 10 μl/ml. Each possibility represents a separate embodiment.

According to some embodiments, Knock-Out Serum Replacement (KSR) may be added to the culture medium.

According to some embodiments, step (a) may be carried out in N2B27 medium. According to some embodiments, step (a) may be carried out is culture medium supplemented with at least one of Knock-out Serum Replacement (KSR), bovine serum albumin (BSA), insulin and BMP4. According to some embodiments, step (a) may be carried out in culture medium supplemented with low concentrations of insulin. According to some embodiments, the low concentrations of insulin are within the range of about 1 to 25 μg/ml insulin, about 1 to 20 μg/ml insulin, about 1 to 15 μg/ml insulin, about 5 to 25 g/ml insulin, or about 5 to 20 μg/ml insulin. Each possibility represents a separate embodiment.

According to some embodiments, the cells are kept in suspension by rotating the plate.

According to some embodiments, the Wnt activator added in step (b) is selected from the group consisting of: Chir99021, Rspo3 and Wnt3a. Each possibility is a separate embodiment.

According to some embodiments, the Wnt activator added in step (b) is in a concentration within the range of about 0.5 to 10 μM, 0.5 to 7 μM, 0.7 to 10 μM, 0.7 to 7 μM, 1 to 7 μM, or 1 to 5 μM. Each possibility represents a separate embodiment.

According to some embodiments, the Wnt activator is added in step (b) about 24 hours following the formation of multiple aggregates in culture. According to some embodiments, the Wnt activator is added in step (b) about 24 hours following the formation of multiple aggregates in suspension.

According to some embodiments, the Wnt activator is removed from the culture medium within about a day after being added to the medium, within 8 to 36 hours after being added within 12 to 36 hours after being added, within 18 to 30 hours after being added, within about 24 hours after being added. Each possibility represents a separate embodiment. In some embodiments, removal of the Wnt activator may be obtained by substituting the culture medium with a similar medium lacking the Wnt activator.

According to some embodiments, the method may further include adding at least one Nodal inhibitor to the culture medium following appearance of the aggregates including said mesodermal and endodermal cells. By supplementing the medium with the at least one Nodal inhibitor, helps repressing the expansion of non-somatic cell populations, such as, definitive endoderm.

According to some embodiments, the Nodal inhibitor is selected from the group consisting of: SB-431542 and SB-505124. Each possibility is a separate embodiment. According to some embodiments, the Nodal inhibitor added in step (b) is in a concentration within the range of 200 nM 50 700 nM, 5 to 15 μM, 5 to 12 μM, 7 to 15 μM, 7 to 12 μM, about 7 μM, or about 10 μM. Each possibility represents a separate embodiment.

According to some embodiments, a scaffolding (extracellular) matrix, or components thereof, are incorporated into the aggregates in the suspension. According to some embodiments, the culture medium includes low concentrations of extracellular matrix (ECM) or components thereof, specifically, less than about 20% vol/vol, less than 15% vol/vol, less than 10% vol/vol, less than 8% vol/vol, less than 5% vol/vol. According to some embodiments, the amount of ECM in the culture medium is within the range of about 1% to 15% vol/vol. According to some embodiments, the amount of ECM in the culture medium is within the range of 1% to 12% vol/vol. According to some embodiments, the amount of ECM in the culture medium is within the range of about 2% to 12% vol/vol. According to some embodiments, the amount of ECM in the culture medium is within the range of about 3% to 13% vol/vol. According to some embodiments, the amount of ECM in the culture medium is within the range of about 2% to 12% vol/vol. According to some embodiments, the amount of ECM in the culture medium is within the range of about 3% to 11% vol/vol. According to some embodiments, the amount of ECM in the culture medium is within the range of about 3% to 10% vol/vol. According to some embodiments, the amount of ECM in the culture medium is within the range of about 4% to 10% vol/vol. According to some embodiments, the amount of ECM in the culture medium is within the range of about 5% to 10% vol/vol.

According to some embodiments, the ECM include Matrigel™. According to some embodiments, the culture medium includes components of the ECM. According to some embodiments, the components of the ECM include, but are not limited to, fibronectin, collagen and/or laminin. According to some embodiments, the components of the ECM include, but are not limited to, fibronectin, collagen and laminin.

According to some embodiments, the addition of ECM or components thereof is performed after observing mesodermal polarization, e.g. by observing aggregates having ovoid morphology. Thus, the protocol is not based on random/constant timing for adding the ECM or its components, since it has been found that the right timing changes from batch to batch, and surprisingly, it is best to add ECM or its components based on observation of the aggregates, wherein the ideal timing is when the aggregates present mesodermal polarization.

“Mesodermal polarization”, also known as morphological symmetry breaking (or just symmetry breaking), is a stage in the formation of the paraxial (or presomitic) mesoderm. During this stage, mesodermal and PSM cells, which initially randomly spread within the aggregate, gradually migrate to a pole of the aggregate. At the same time, the aggregate morphology changes from spherical to ovoid. Mesodermal polarization may be identified by the ovoid shape assumed by the aggregates or by polar location of the mesodermal or PSM cells, as detailed below.

According to some embodiments, the mesodermal polarization is identified by the aggregates assuming an ovoid morphology as determined by an aspect ratio of the aggregate being 1.2 or higher. Accordingly, step (d) includes adding extracellular matrix or components thereof when the aspect ratio of the aggregates is 1.2 or higher (see, e.g., FIG. 2A, day 4.5).

The term “aspect ratio”, as used herein with reference to the aggregates, is defined as [length of long axis]/[length of short axis]. These are computed by image analysis of the brightfield (non-fluorescent) microscopy channel. Aspect ratio ranges between 1 (perfect circle) to infinity (a thin line).

According to some embodiments, the mesodermal polarization is identified by staining mesodermal or PSM cells in the aggregates including the mesodermal and endodermal cells with a suitable marker to identify the mesodermal or PSM cells accumulating at poles of the aggregates.

According to some embodiments, the suitable marker is Mesogenin1 (Msgn1), T-box transcription factor 6 (Tbx6), or Brachyury (T or TbxT).

Following the addition of ECM or components thereof, paraxial mesoderm is formed. According to some embodiments, the ECM or components thereof are removed from the culture medium following formation of the paraxial mesoderm within the aggregates. Removal of ECM or its components from the culture medium confers an additional advantage to the disclosed protocol: it reduces the overall cost of the entire protocol while not compromising on the desired outcome.

According to some embodiments, there is also the option to maintain in the medium the ECM or components thereof.

According to some embodiments, the method may further include adding at least one Wnt activator and/or at least one BMP inhibitor to the medium in step (d). Thus, according to some embodiments, the medium in step (d) may further include at least one Wnt activator and/or at least one BMP inhibitor. Supplementing the medium in step (d), namely, a medium including the ECM or components thereof, with Wnt activator(s) and/or BMP inhibitor(s) is beneficial as it can improve the yield of somite formation. Furthermore, the addition of Wnt activator(s) and/or BMP inhibitor(s) to the medium at this stage reduces the overall viscosity of the medium thereby facilitating growth in a mixed suspension condition, which is especially suitable for industrial scale bioreactors.

According to some embodiments, the at least one Wnt activator is selected from Chir99201 and Rspo3. Each possibility represents a separate embodiment.

According to some embodiments, the at least one BMP inhibitor is selected from LDN193189 and Noggin. Each possibility represents a separate embodiment.

According to some embodiments, a plurality of organoids are embedded at the end of the differentiation process, into a scaffolding matrix.

The terms “aggregates”, “3D organoids” and “organoids” as used herein, are interchangeable.

According to some embodiments, there is provided a method for generating myogenic progenitors, the method including the steps recited above for obtaining somites and further includes step (f) adding one or more growth factors, the concentration of which is below about 30 ng/ml, thereby obtaining myogenic progenitors.

According to some embodiments, the one or more growth factors include, but are not limited to, HGF, IGF and FGF2, wherein the concentration on the one or more growth factor is within the range of about 1 to 25 ng/ml. According to some embodiments, the medium of step (f) is further supplemented with at least one of compound selected from: BMP inhibitor and Wnt activator.

According to some embodiments, the one or more growth factors may include HGF. According to some embodiments, the concentration of HGF added to the medium of step (f) is within the range of about 5 to 15 μl/ml, 5 to 13 μl/ml, 7 to 13 μl/ml, 7 to 12 μl/ml, 5 to 12 μl/ml, about 7 μl/ml, or about 10 μl/ml. Each possibility represents a separate embodiment.

According to some embodiments, the one or more growth factors may include IGF. According to some embodiments, the concentration of IGF added to the medium of step (f) is within the range of about 0.5 to 5 μl/ml, 0.5 to 3 μl/ml, 0.7 to 3 μl/ml, 0.7 to 2.5 μl/ml, 1 to 2.5 μl/ml, about 2.5 μl/ml, or about 2 μl/ml. Each possibility represents a separate embodiment.

According to some embodiments, the one or more growth factors may include FGF2. According to some embodiments, the concentration of FGF2 added to the medium of step (f) may be within the range of about 10 to 25 μl/ml, 12 to 25 μl/ml, 12 to 23 μl/ml, 15 to 25 μl/ml, 15 to 23 μl/ml, about 25 μl/ml, or about 20 μl/ml. Each possibility represents a separate embodiment.

According to some embodiments, the obtained myogenic progenitors include myoblasts, myocytes and satellite cells.

Maturation of muscle cells has been demonstrated to depend on anchoring of the cells on (semi-)rigid surfaces, likely mimicking anchoring to bone structures. Accordingly, the method disclosed herein may further include the step of embedding the late-stage organoids in edible matrix, in an attempt to generate anchor points of the maturation of myocytes. Optional matrices include, but are not limited to, mycelium, alginate and cellulose (e.g. from decellularized apples).

According to some embodiments, the method further including monitoring differentiation in the suspended 3D-organoids.

According to some embodiments, the monitoring may be carried out using at least one technique selected from the group consisting of: high throughput two-photon 3D imaging, live imaging with GFP-tagged developmental markers (such as Msgn1, Tbx6, Brachyury (T or TbxT), Pax3, Pax7), and immunostaining against marker proteins.

Imaging is also used to monitor the cell composition at the different phases of the process for the purpose of identifying efficiency bottlenecks in terms of the fraction of muscle lineage cells in the organoid.

According to some embodiments, the method may further include characterizing the cell populations in the 3D-organoids at different time points.

According to some embodiments, the method may further include characterizing the spatial organization of the cell populations in the 3D-organoids at different time points.

According to some embodiments, characterizing the cell populations in the 3D organoids may be carried out by various methods, including, but not limited to, RT-PCR and FISH.

According to some embodiments, the method may further include nutritional profiling the 3D organoids. For nutritional profiling various approaches can be applied, such as, GC, HPLC, and GC-MS with cold EI.

According to some embodiments, the 3D organoids are in a format selected from the group consisting of hanging drops, rotating suspension, free suspension, patterned microwells and high-throughput 96-wells.

According to some embodiments, there are provided cultured somites and cultured tissues including mature muscle progenitor cells or mature muscle cells, obtained by the protocols disclosed herein.

In the description and claims of the application, the words “include” and “have”, and forms thereof, are not limited to members in a list with which the words may be associated.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLES

### Example 1: Protocol for Generation of Somites and Myogenic Progenitor

The 3D differentiation model disclosed herein forms basis for industry-level scaleup processes and adaptation to other edible species. A full suspension, cell-cluster based protocol as disclosed herein can be incorporated in bioreactors of unlimited size. Moreover, the protocol is serum free, and does not involve genetic modifications, rendering it suitable for industrial processing.

The following protocol provides myogenic progenitor in several days. The protocol includes the following steps:

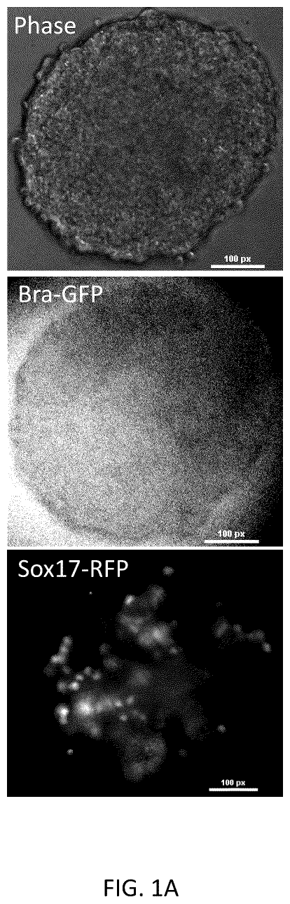

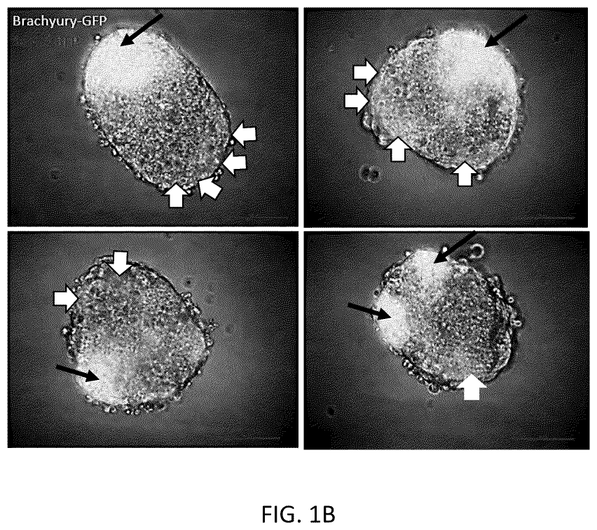

ESC aggregate formation (Step 1): aggregate spheres of 50-300 ESCs were obtained by seeding the ESCs in an N2B27 or NDIFF medium (e.g. at an ULA u-bottom plate; FIG. 1A) (corresponding to step (a) of the disclosed method). The medium may be supplemented with one or more of Insulin (e.g. 10 μl/ml), transferrin-selenium (e.g. 1%) and BMP4 (e.g. 10 ng/ml). Aggregate spheres of 20-300 ESCs were also obtained by culturing the ESCs at a density of 10K-30K cells/ml in N2B27 medium supplemented with 1-5% Knock-out Serum Replacement (KSR), 10-30 μg/ml bovine serum albumin (BSA), low concentrations of 5-15 μg/ml insulin and 5-15 ng/ml BMP4 (FIG. 1B). Preferably, the cells should be kept in suspension, e.g., by rotating the plate at speed of 100-300 rpm.

Germ layers formation (Step 2): this step was carried out about one day after multiple aggregates were formed, or after multiple floating cell-aggregate spheres with diameter between 50-200 μm were generated, in each well. In this step the Wnt pathway was activated by adding to the culture medium Wnt activator Chir99021 (1-5 μM) or Rspo3 (10-30 ng/ml) or Wnt3a (50-300 ng/ml) for approximately 12 to 24 hours (corresponding to step (b) of the disclosed method). Thereafter, the medium containing the Wnt activator was removed and replaced with a fresh medium, devoid of Wnt activator (corresponding to step (c) of the disclosed method).

The addition of Wnt activator pushes the differentiation towards the mesodermal lineage, and as a result, at the end of this step, mesodermal cells (marked by Brachyury (T or TbxT)-GFP; e.g. FIG. 1B-thin arrows) and endodermal cells (marked with PSM cells RFP; FIG. 1B, wide arrows) were visible in the aggregate, as shown in FIGS. 1A and 1B. It is noted that the morphology of aggregates obtained at the end of this step and germ layers formation, is typically an ovoid morphology as can be seen in FIG. 2A (day 4.5).

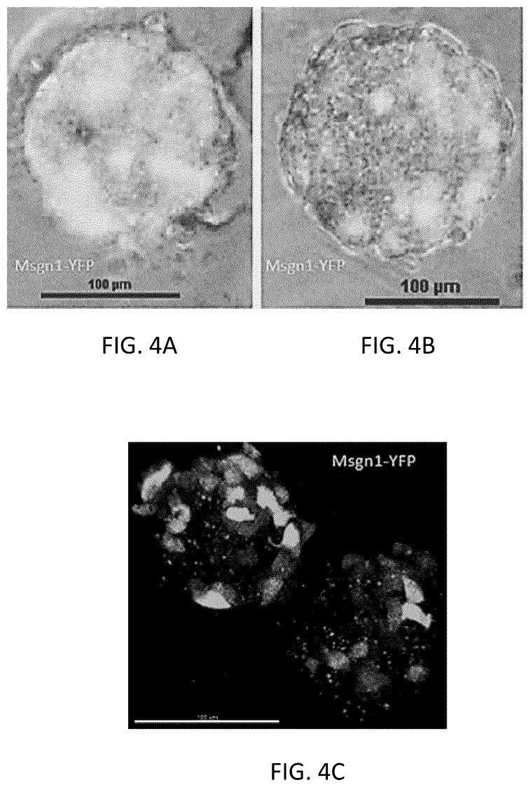

Paraxial mesoderm (PSM, also termed presomitic mesoderm) formation (Step 3): PSM cells were visible within 1-2 days after the first mesodermal cells (Brachyury-positive cells) appeared, and could be identified using the marker Mesogenin1 (Msgn1) (FIGS. 2A-2E and FIGS. 4A-4C). As shown in FIG. 2A, the cells initially spread randomly in the aggregate (FIG. 2A, left panel, Day 4) and gradually concentrate on one location of the aggregate. It is noted that Tbx6 may be used as an alternative marker for Msgn1.

One up to two days after the first PSM cells appear, the morphology of the aggregates changed from circular, round spheres, to ovoid, in parallel to the maturation of the paraxial mesoderm (e.g. FIG. 2A middle panel, FIG. 2C and FIG. 2F). The PSM cells at this stage migrated and aggregated in one (or several) poles (as part of the mesodermal polarization phase) within the aggregate (e.g. FIGS. 2A and 2D, bright regions).

Once the aggregates morphology changes from round sphere to ovoid morphology low percentage of extra cellular matrix (ECM), such as 5-10% Matrigel, or one of the following ECM components (fibronectin, collagen, and/or laminin) was added (corresponding to step (d) of the disclosed method). Optionally, one or more of the following, may be added to the medium 12-24 hours after the onset of the mesodermal cells:
(a) Nodal inhibitor (e.g. 5-15 μM SB-431542).
(b) Wnt activator (e.g. Chir99201 or Rspo3) at high concentration, typically within the range of 3 μM to 9 μM, and 20-60 ng/ml, respectively.
(c) BMP inhibitor (e.g. LDN193189 or Noggin).

The aggregates are then incubated for 10-48 hours with the ECM components (corresponding to step (e) of the disclosed method).

The ovoid morphology may be determined by calculating the aspect ratio of the aggregates, which should be 1.2 or higher. Aspect ratio may be computed by image analysis and is defined as [length of the long axis]/[length of the short axis].

Alternatively, the time point for adding ECM or components thereof may be determined by identifying PSM cells presenting a Mesogenin1 (Msgn1) or a T-box transcription factor 6 (Tbx6) marker, or mesodermal cells presenting a Brachyury (T or TbxT) marker, in the aggregates including the mesodermal and endodermal cells, and adding extracellular matrix or components thereof when these mesodermal or PSM cells accumulate at poles of the aggregates, to obtain aggregates including paraxial mesoderm.

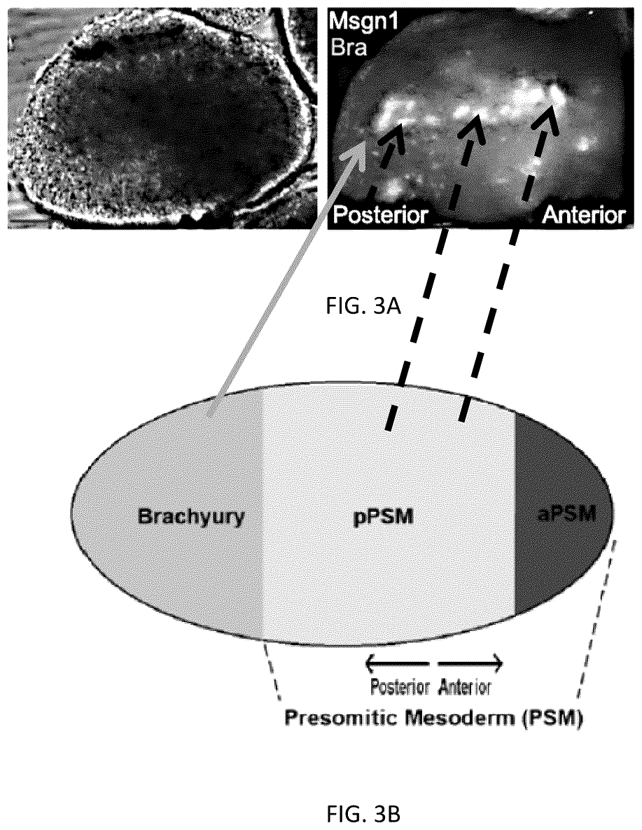

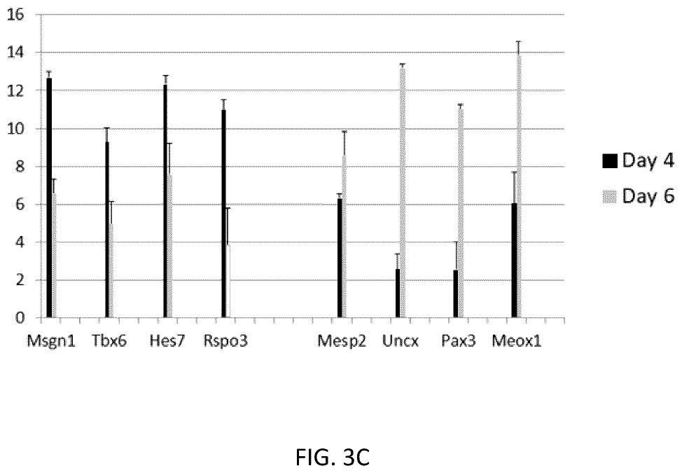

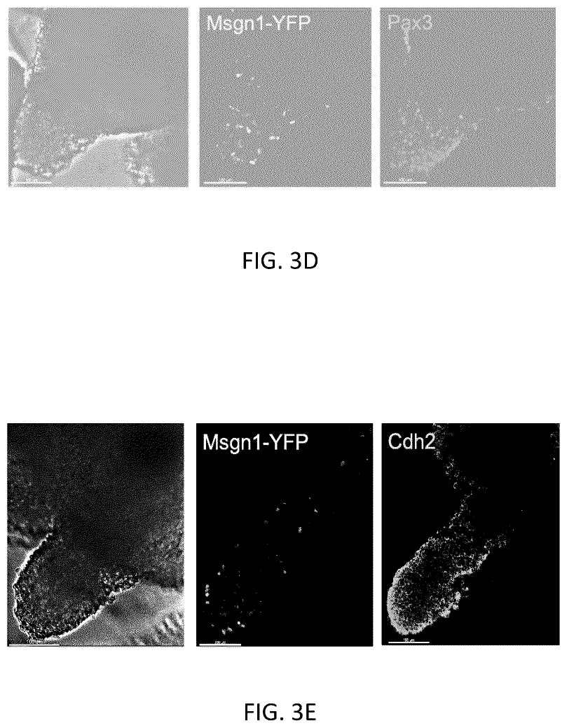

PSM maturation and formation of anterior presomitic mesoderm (aPSM) cells, that are Pax3-positive, take place in parallel to the aforementioned mesodermal polarization. The aPSM cells were generated at the anterior pole of the PSM domain within the aggregate (e.g. FIGS. 3A and 3B). These cells are capable of giving rise to somites and later on to myogenic progenitors.

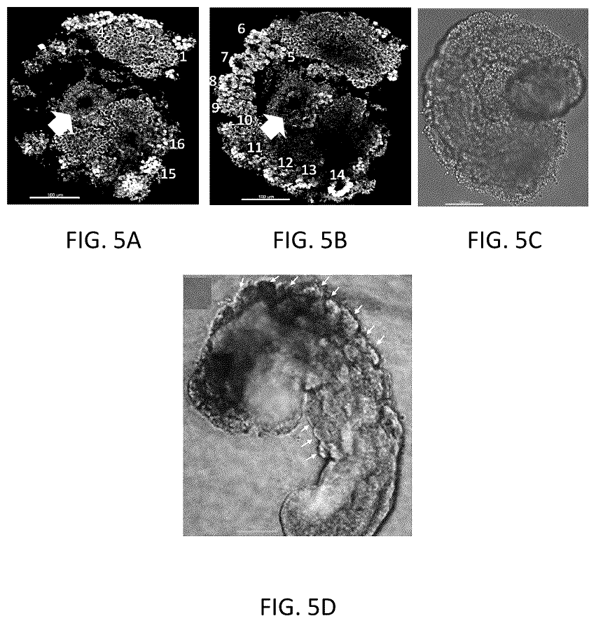

Somite formation and axis elongation (Step 4): ECM (or ECM components) were removed from the medium about 1 or 2 days after the formed paraxial mesoderm aggregates were fully elongated and included somite-like structures (which are numbered and pointed at in FIGS. 5A-5B and 5D, respectively). It is noted that ECM removal is optional. It is further noted that the morphological change from round spheres (e.g., FIG. 1A), through ovoid shape (e.g. FIG. 2C) into the elongated shape shown in FIGS. 5A-5D indicates that the protocol disclosed herein resembles embryonic development.

Myogenic progenitor formation (Step 5): about 1-2 days after the formation of somites-like structures low concentrations of one or more of the following growth factors (GFs) were added to the medium: 10 ng/ml HGF, 2 ng/ml IGF, 20 ng/ml FGF2 (step (f) of the disclosed method). Optionally, the GFs are added alone or in combination with one or more of BMP inhibitor, Wnt activator and ECM as mentioned in previous steps.

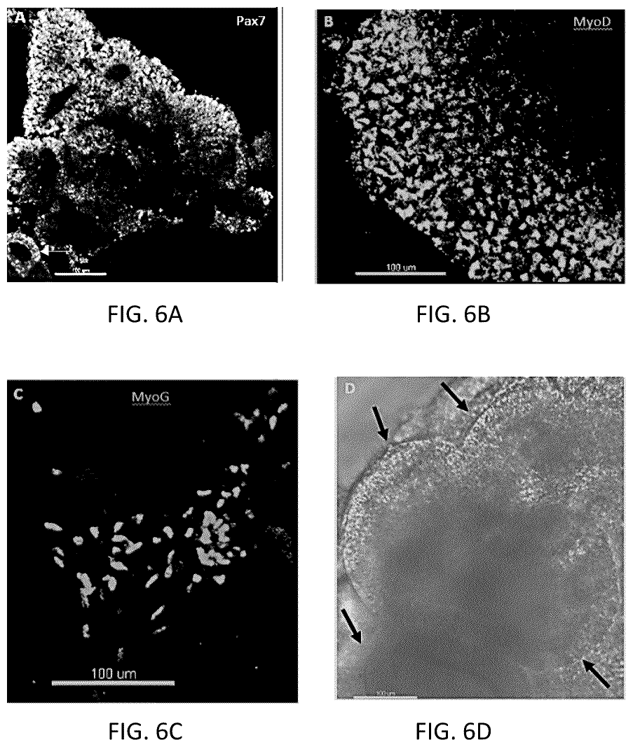

At the end of this step, about 1-2 days after the addition of GFs, myogenic progenitors, such as myoblasts, myocytes and others were visible in the mature somites (FIGS. 6A-6D).

The culture medium was refreshed every other day throughout the protocol.

### Example 2: Spatial Expression of Stage-Specific Markers for Paraxial Mesoderm Differentiation

The molecular and cellular properties of the in-vitro model were compared to embryo. This comparison is crucial as neighboring cell layers provide essential signaling and mechanical support for muscle tissue formation.

To this end, the spatial expression of stage-specific markers for paraxial mesoderm differentiation (Brachyury/T, Msgn1, Pax3, Mesp2, Meox1), somites (Uncx, Tbx18), muscle progenitors (Pax7), Myocytes (MyoG) and mature muscle cells (fast MHC) were characterized. This was done using existing fluorescent live marker cell lines such as, Brachyury, Msgn1, Pax3, Pax7, MyoG, or immunostaining with marker-specific antibodies.

A spatial mRNA expression map at different stages, for an extended set of genes pre-selected for each stage, is also obtained using fluorescent in-situ hybridization (FISH). This method is essential for genes for which there are no validated antibodies. This becomes critical in non-model organisms such as cow or chicken, where antibody availability is particularly limited.

### Example 3: Somatic Mesoderm Generation from Fish Embryonic Stem Cells

Denis (seabream) pluripotent cells were seeded in 96ULA plates or in 15 mL tubes on a rotating device, in a growth culture medium. 2 μM of CHIR was added at the time of forming aggregates (about 24 hours after seeding), and the medium was replaced with a medium without CHIR after 48 h. The added replacement medium included ECM components.

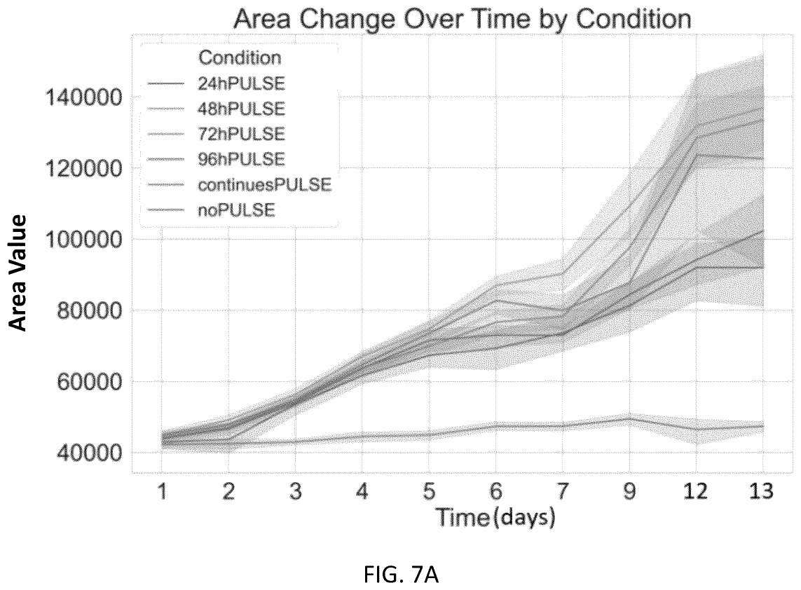

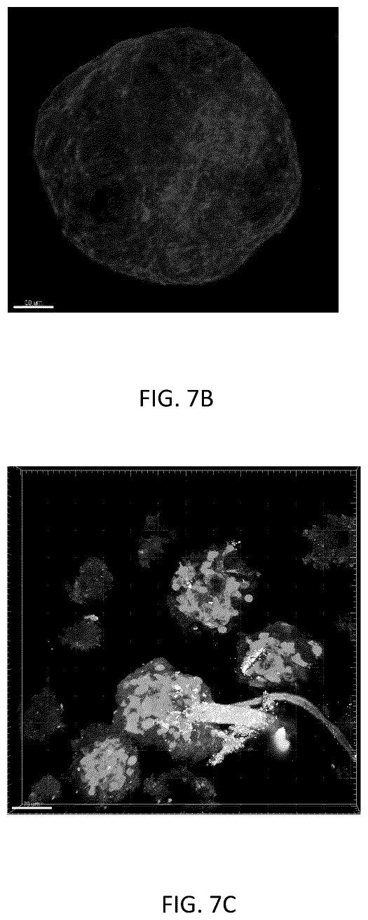

FIG. 7A shows aggregate surface area between day 1 and day 13 under different conditions, demonstrating fastest cell growth was under the above-described conditions (48 h pulse). 24 days after seeding, samples were stained with phalloidin rhodamine (an F-actin marker), revealing fibrous staining patterns in some regions of the aggregates (FIG. 7B), indicating skeletal muscle formation.

To further investigate muscle differentiation, two-month-old aggregates were stained for titin, a muscle-specific structural marker. Titin-positive staining was observed in some aggregates (FIG. 7C, in green), indicating muscle differentiation. The presence of skeletal muscle fibers indicates the generation of somatic mesoderm, from which muscle progenitor cells arise.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1. A method for generating somites, the method comprising:
(a) suspending embryonic stem cells in culture medium to obtain spheric aggregates;
(b) adding to the culture medium at least one Wnt activator to obtain aggregates comprising mesodermal and endodermal cells;
(c) removing the Wnt activator;
(d) observing the aggregates comprising the mesodermal and endodermal cells, and adding extracellular matrix or components thereof after identifying mesodermal polarization in the aggregates, to obtain aggregates comprising paraxial mesoderm; and
(e) incubating the aggregates of step (d), for 10 to 48 hours, thereby obtaining aggregates comprising a plurality of somites
wherein the embryonic stem cells are selected from the group consisting of human, mouse, and fish embryonic stem cells.

2. The method according to claim 1, further comprising adding to the culture medium of step (a) one or more of insulin, Knock-out Serum Replacement, transferrin-selenium and BMP4.

3. The method according to claim 1, wherein the Wnt activator is selected from the group consisting of: Chir99021, Wnt3a and Rspo3.

4. The method according to claim 1, wherein said adding to the culture medium at least one Wnt activator is carried out within 12 to 36 hours after obtaining said spheric aggregates.

5. The method according to claim 1, wherein said removing the Wnt activator is carried out within the range of 8 to 36 hours.

6. The method according to claim 1, further comprising adding at least one Nodal inhibitor to the culture medium following appearance of the aggregates comprising said mesodermal and endodermal cells.

7. The method according to claim 1, wherein the amount of the extracellular matrix or components thereof is within the range of 1 to 15% vol/vol.

8. The method according to claim 1, wherein the mesodermal polarization is identified by the aggregates assuming an ovoid morphology as determined by an aspect ratio of the aggregates being 1.2 or higher.

9. The method according to claim 1, wherein the mesodermal polarization is identified by staining mesodermal or presomitic mesoderm (PSM) cells in the aggregates comprising the mesodermal and endodermal cells with a suitable marker, to identify the mesodermal or PSM cells accumulating at poles of the aggregates.

10. The method according to claim 1, wherein the embryonic stem cells are from teleost fish.

11. The method according to claim 1, wherein the embryonic stem cells are from seabream or eel.

12. The method according to claim 1, wherein the embryonic stem cells are from seabream.

13. The method according to claim 1, further comprising adding in step (d) at least one compound selected from the group consisting of: Wnt activator and BMP inhibitor.

14. The method according to claim 1, further comprising removing the extracellular matrix or components thereof, following said obtaining the plurality of somites.

15. The method according to claim 1, further comprising a step (f), comprising adding one or more growth factors to the medium in step (e), the concentration of which is below 30 ng/ml, thereby generating myogenic progenitors.

16. The method according to claim 15, wherein the one or more growth factors include HGF, IGF and FGF2.

17. The method according to claim 15, wherein the concentration of each growth factor is within the range of 1 to 25 ng/ml.

18. The method according to claim 15, wherein the one or more growth factors is IGF, and the concentration of IGF is within the range of 1 to 5 ng/ml.

19. The method according to claim 15, wherein the myogenic progenitors comprise myoblasts and myocytes.
